(19) United States
(12) Patent Application Publication
(10) Pub. No.: US 2022/0347326 A1
(43) Pub. Date: Nov. 3, 2022

(54) PORTABLE STERILIZATION DEVICE

(71) Applicant: OXTI CORPORATION, New Taipei City (TW)

(72) Inventor: Chih-Feng Ho, New Taipei City (TW)

(21) Appl. No.: 17/243,517

(22) Filed: Apr. 28, 2021

(51) Int. Cl. A61L 2/10

(52) U.S. Cl. CPC A61L 2/10; A61L 2202/11; A61L 2202/16

(57) ABSTRACT

A portable sterilization device, includes a box body, configured with an accommodation space; at least one lamp, configured inside the box body; and at least one shroud, configured inside the box body, and positioned relatively above the lamp. With the above structure, items to be sterilized are placed inside the accommodation space of the box body, thereby preventing the items from being in contact with other pollution sources. The shrouds inside the box body can shield the light emitted from the lamps so as to avoid light directly irradiating human bodies. The lamp is used to emit light for sterilization and disinfection, and the light for example is ultraviolet light capable of effectively carrying out sterilization on the items inside the box body. At the same time, the overall structure is simple, light and easy to carry.

### (a) TECHNICAL FIELD OF THE INVENTION

The present invention relates to a portable sterilization device, and more particularly to a box-shaped portable sterilization device.

### (b) DESCRIPTION OF THE PRIOR ART

With the change of human life style, the global climate change changes the transmission route and distribution area of infectious diseases. For example, climate change having changed the habitat of these virus host animals, humans having invaded more and more primitive ecosystems, the frequency and speed of human migration, and overpopulation are all reasons for the rapid spread of infectious diseases. Viruses and pathogenic microorganisms continue to adapt and mutate, causing every kind of emerging infectious diseases and re-emerging infectious diseases to happen incessantly; from early measles, smallpox, malaria, influenza to the recent severe acute respiratory syndrome (SARS), new type A influenza virus (H1N1), Middle East respiratory syndrome (MERS-CoV) and new coronavirus pneumonia (COVID-19), the currently known infectious transmission of such infectious diseases is mainly spread by direct contact with viral secretions or droplets.

Patients cause viruses to attach to various items such as bathroom toiletries due to coughing, sneezing or contact with respiratory secretions. For example, Chinese Utility Patent No. CN2426360Y, titled “Dedicated disinfection cabinet for toiletries”, discloses a disinfection cabinet including a cabinet body with shelves inside and cabinet door, ozone generator fixed on the inner surface of the cabinet body, and time switch installed on the outer surface of the cabinet. The cabinet door is a built-in structure, and a sealing tape is fixed on the inner surface of the cabinet door corresponding to the front edge of the cabinet body; shelves and cross bars are configured on the inner surface of the cabinet door, and several dental bayonets on each shelf. However, such kinds of disinfection products have more functions, but the structure is more complicated and not easy to carry.

### SUMMARY OF THE INVENTION

To overcome the above disadvantages, the present invention proposes a portable sterilization device, including: a box body, configured with an accommodation space; at least one lamp, configured inside the box body; and at least one shroud, configured inside the box body, and positioned relatively above the lamp.

Preferably, a concave portion is configured inside the box body.

Preferably, the lamp is provided with a signal receiver.

Preferably, the lamp is provided with a power wire.

Preferably, the at least one shroud is inclinedly configured inside the box body.

With the above structure, items to be sterilized are placed inside the accommodation space of the box body, thereby preventing the items from being in contact with other pollution sources. The shrouds inside the box body can shield the light emitted from the lamps so as to avoid light directly irradiating human bodies. The lamp is used to emit light for sterilization and disinfection, and the light for example is ultraviolet light capable of effectively carrying out sterilization on the items inside the box body. At the same time, the overall structure is simple, light and easy to carry. In addition, the lamp is further provided with the signal receiver so as to receive a wireless signal, thereby being able to remotely control light emission. The concave portion 112 is further configured inside the box body 11 for placing the box cover 113. The shroud is inclinedly configured inside the box body 11, more effectively shielding the light emitted from the lamp 12, allowing the light to be not easy to directly irradiate human bodies.

### BRIEF DESCRIPTION OF THE DRAWINGS

FIG. 1 is an exploded view of a portable sterilization device of the present invention;

FIG. 2 is a perspective view of the portable sterilization device of the present invention;

FIG. 3 shows the portable sterilization device being used on a toilet water tank;

FIG. 4 shows the portable sterilization device being used on a washbasin; and

FIG. 5 shows the portable sterilization device being used inside a wardrobe.

### DETAILED DESCRIPTION OF THE PREFERRED EMBODIMENTS

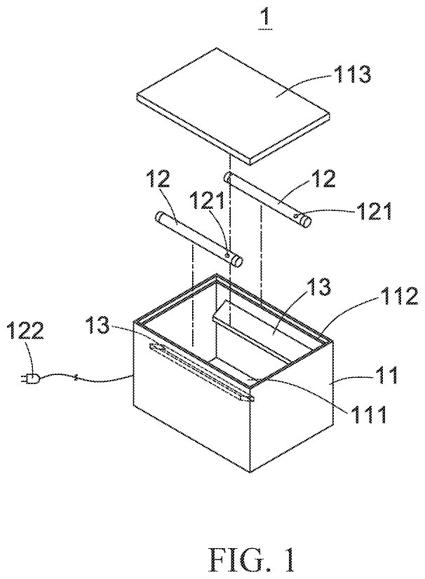

Referring to FIG. 1, a portable sterilization device of the present invention includes a box body 11, at least one lamp 12 and at least one shroud 13.

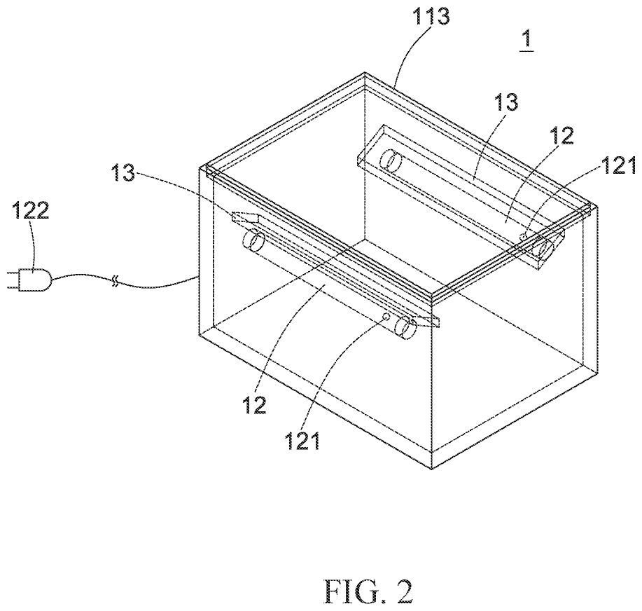

Referring to FIG. 2, the box body 11 has at least one side plate and bottom plate defining an accommodation space 111 of the box body 11, allowing the box body 11 to be configured with the accommodation space 111.

The inside of the box body 11 is provided with a concave part 112, which is formed on the side plates of the box body 11 and is used to install and place a box cover 113, which can prevent water from splashing into the inside of the box body 11 or water vapor from dripping into the inside of the box body 11 after condensation.

The at least one lamp 12 adapted to emit light for disinfection and sterilization includes components such as a light source, control circuit board, where the light may be ultraviolet light. The lamp 12 may for example be a UV-B ultraviolet lamp, UV-C ultraviolet lamp, mercury lamp, metal halide lamp or xenon lamp, but the present invention is not so limited. The lamp body 12 is further configured with a signal receiver 121 adapted to receive a wireless signal to enable the light-emitting of the lamp 12 to be remotely controlled. Except a built-in battery, the lamp 12 is further provided with a power wire 122 adapted to be in connection with an external power source, allowing the lamp 12 to obtain needed power.

The at least one shroud 13 is configured inside the box body 11 and positioned relatively above the lamp 12. The shroud 13 can shield the light emitted from the lamp 12, thereby avoid the light emitted from the shroud 13 directly irradiating human bodies, thereby preventing the human bodies from being harmed. Furthermore, the shroud 13 may be inclinedly arranged inside the box body 11, and further provided with a mirror with reflection, making the light emitted from the lamp 12 more concentrated on the bottom of the box body 11.

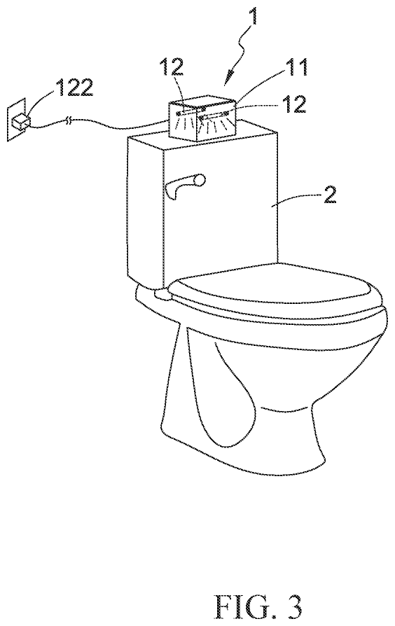

Referring to FIG. 3, the portable sterilization device 1 can be used on a toilet tank 2 of a bathroom, and the power wire 122 of the lamp 12 is plugged into a power plug, allowing the lamp 12 to emit light to sterilize and disinfect items to be sterilized inside the box body 11.

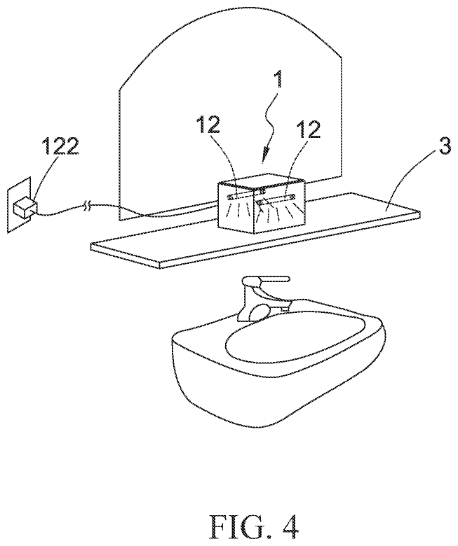

Referring to FIG. 4, the portable sterilization device 1 can be used on bathroom washbasin shelf 3, and the power wire 122 of the lamp 12 is plugged into a power plug, allowing the lamp 12 to sterilize and disinfect the items to be sterilized inside the box body 11.

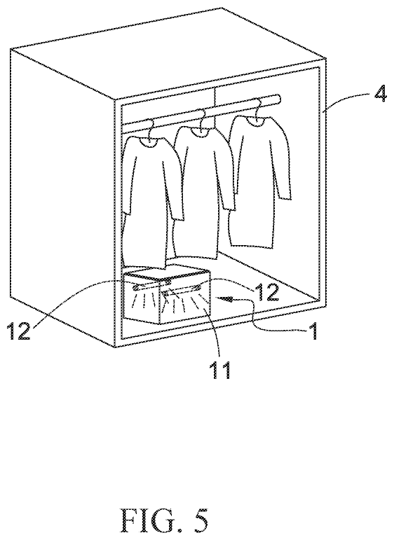

Referring to FIG. 5, the portable sterilization device 1 can be used inside a wardrobe, and the built-in battery can be used to supply power to the lamp 12, allowing the lamp 12 to emit light to sterilize and disinfect the items to be sterilized inside the box body 11.

Conclusively, with the above structure, items to be sterilized such as toiletries are placed inside the accommodation space 111 of the box body 11, thereby preventing the items from being in contact with other pollution sources. The shrouds 13 inside the box body 11 can shield the light emitted from the lamps 12 so as to avoid light directly irradiating human bodies. The lamp 12 is used to emit light for sterilization and disinfection, and the light for example is ultraviolet light capable of effectively carrying out sterilization on the items inside the box body 11. At the same time, the overall structure is simple, light and easy to carry. In addition, the lamp 12 is further provided with the signal receiver 121 so as to receive a wireless signal, thereby being able to remotely control light emission. The concave portion 112 is further configured inside the box body 11 for placing the box cover 113. The shroud 13 is inclinedly configured inside the box body 11, more effectively shielding the light emitted from the lamp 12, allowing the light to be not easy to directly irradiate human bodies.

I claim:

1. A portable sterilization device, comprising:
a box body, configured with an accommodation space;
at least one lamp, configured inside said box body; and
at least one shroud, configured inside said box body, and positioned relatively above said lamp.

2. The device according to claim 1, wherein a concave portion is configured inside said box body.

3. The device according to claim 1, wherein said lamp is provided with a signal receiver.

4. The device according to claim 1, wherein said lamp is provided with a power wire.

5. The device according to claim 1, wherein said at least one shroud is inclinedly configured inside said box body.
